(19) United States
(12) Patent Application Publication
(10) Pub. No.: US 2010/0255485 A1
(43) Pub. Date: Oct. 7, 2010

(54) BIOMARKERS FOR THE ONSET OF NEURODEGENERATIVE DISEASES

(76) Inventors: Pico Caroni, Muttenz (CH); Smita Saxena, Basel (CH)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/744,385

(22) PCT Filed: Nov. 27, 2008
(86) PCT No.: PCT/EP08/66291

§ 371 Date: May 24, 2010

(30) Foreign Application Priority Data
Nov. 28, 2007 (EP) 07121792.1

(51) Int. Cl. C12Q 1/68

(52) U.S. Cl. 435/6

(57) ABSTRACT

The present invention provides a method of predicting the imminent degeneration of motoneurons in a subject, said method comprising assessing in at least one motoneuron of said subject the expression of at least one particular gene, wherein an at least two-fold upregulation of the expression of the assessed genes is indicative of the imminent degeneration of motoneurons and/or of the imminent onset of a neurodegenerative disease. Kits therefor are also provided.

### FIELD OF THE INVENTION

The present invention provides a method of predicting the imminent degeneration of motoneurons in the context of neurodegenerative diseases.

### BACKGROUND OF THE INVENTION

In subjects having neurodegenerative disease neurons of the brain and spinal cord are lost. Examples of neurodegenerative diseases include Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis and Charcot-Marie-Tooth disease.

Amyotrophic lateral sclerosis (ALS) is a fatal neurodegenerative disease that affects selectively motoneurons in the central nervous system. Most ALS patients die within five years of onset, and the mechanisms of the onset of the disease, as well as of its progression are poorly understood.

The present inventors have previously examined two mouse models of motoneuron (MN) disease (SOD1(G93A), SOD1(G85R)), and found that axons of fast-fatiguable (FF) MNs are affected synchronously, long before symptoms; fast-fatigue-resistant (FR) MN axons are affected at the onset of symptoms, and axons of slow (S) MNs are resistant (Pun et al., 2006, Nature Neuroscience, 9(3):408-419).

One of the main aspects in understanding the initiation and progression of a neurodegenerative disease such as in ALS is to elucidate mechanisms that underlie or predispose a particular neuron (in this case a MN) to selective vulnerability.

There is hence a need in the art for biomarkers that would be able to predict the imminent onset of a neurodegenerative disease.

### SUMMARY OF THE INVENTION

The present inventors have therefore selectively labeled vulnerable motoneurons innervating the purely FF subcompartment of lateral gastrocnemius, and resistant motoneurons innervating the soleus muscle at different times in wildtype and SOD (G93A) and G85R mice in order to study gene expression alterations in MNs before disease onset, at onset and during disease progression. The present inventors analyzed the gene expression profiles of 8-10 labeled MNs at defined time in disease. This approach has allowed the present inventors to investigate disease progression in vulnerable and resistant MNs in a “longitudinal” manner, i.e. comparing values at different time-points before, at onset and during progression of the disease

Using the above mentioned approach, the present inventors have surprisingly shown that 28 genes which mark the acute phase of the disease and whose alteration in expression specifically precedes the first macroscopically detectable symptoms are upregulated initially in the most vulnerable FF MN axons, and later in the FR MN axons, which degenerate after FF MNs in disease. The inventors have further shown that upregulation of the 28 genes reflects triggering of an Unfolded Protein Response (UPR) selectively in the vulnerable MNs. The present inventors therefore provide a method for the prediction of the imminent degeneration of motoneurons in a subject, said method comprising assessing in at least one motoneuron of said subject the expression of at least one gene selected from the group consisting of elF2a, Atf3, Laptm5, GADD45, LAPTM, BiP, Grn, and Nrn1, wherein an at least two-fold upregulation of the expression of the assessed gene is indicative of the imminent degeneration of motoneurons and/or of the imminent onset of a neurodegenerative disease.

In one embodiment of the invention, the expression of at least one second gene selected from the group consisting of Hex B, Apeg1, Csf1r, Cth, Cyp3a11, Ddit4l, Gas5, Gbp2, Jag1, Kcnc2, Lzp-s, Nfe2l3, Ndrg1, Nrxn3, Pck2, Rex3, Slc7a3, Syt 7, Tyrobp, Zcchc12 and Zic1, is also assessed, wherein an at least two-fold upregulation of the expression of the assessed genes is indicative of the imminent degeneration of motoneurons and/or of the imminent onset of a neurodegenerative disease.

In one embodiment of the invention, the expression of at least Atf3, Laptm5, elF2a, GADD45, is assessed.

In some embodiments of the methods of the invention, an increase of the expression of at least one, two, three, four, five, six, seven or eight of these genes is indicative of the disease mediated stress response and imminent degeneration of motoneurons.

In yet another preferred embodiment of the invention, the expression of at least elF2a is assessed.

In another embodiment, the method of the invention further comprises assessing the presence of, or measuring the concentration of, a product of at least one gene selected from the group consisting of Ctss, C1qb, Igtb, Ifih1 and Tgfbi in a body fluid obtained from said subject.

In yet another embodiment of the method of the invention, said method further comprises assessing in at least one motoneuron of said subject the expression of at least one gene selected from the group consisting of Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd, wherein an at least two-fold downregulation of the expression of at least one of the assessed genes is indicative of the imminent degeneration of motoneurons and/or of the imminent onset of a neurodegenerative disease.

In some embodiments of the invention, the motoneurons of the subject present an accumulation of poly-ubiquitin, as compared to motoneurons of a control subject, or control population, and/or an increase in ribosomal protein S6 phosphorylation, as compared to motoneurons of a control subject, or control population.

Another aspect of the invention is the provision of a kit for predicting the imminent degeneration of motoneurons in a subject, said kit comprising means to assess in at least one motoneuron of said subject the expression of at least one gene selected from the group consisting of elF2a, Atf3, Laptm5, GADD45, LAPTM, BiP, Grn, Nrn1, Hex B, Apeg1, Csf1r, Cth, Cyp3a11, Ddit4l, Gas5, Gbp2, Jag1, Kcnc2, Lzp-s, Nfe2l3, Ndrg1, Nrxn3, Pck2, Rex3, Slc7a3, Syt 7, Tyrobp, Zcchc12 and Zic1.

In an alternative embodiment of this aspect of the invention, the kit further comprises means to assess the presence and or concentration of a product of at least one gene selected from the group consisting of Ctss, C1qb, Igtb, Ifih1 and Tgfbi in a body fluid obtained from said subject.

In yet another embodiment of this aspect of the invention, the kit further comprises means to assess in at least one motoneuron of said subject the expression of at least one gene selected from the group consisting of Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd.

### DESCRIPTION OF THE FIGURES

FIG. 1: Potential biomarkers selected from gene microarrays expressed as fold change. Genes showing a 2 fold and above changes in raw expression value between P32 and P37, p≦0.05.

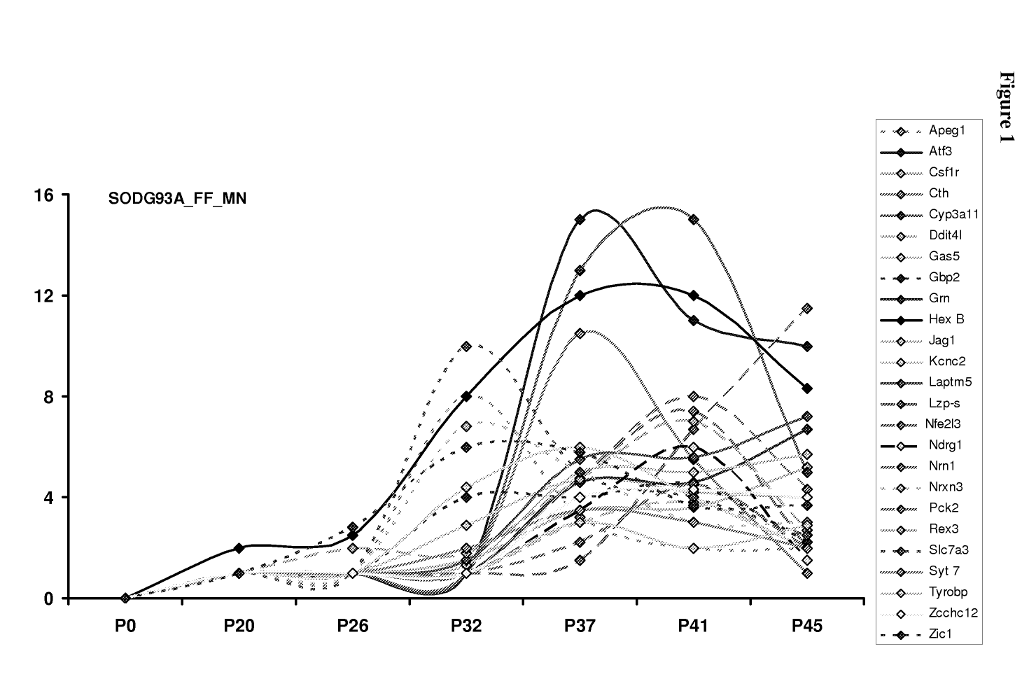

FIG. 2: Upregulation of UPR and stress genes, and downregulation of UPS genes in VUL MNs from P32 on. Analysis based on Ingenuity software. Data from 3 mice each; the analysis is based on net changes (up or down) in transcript levels. Note how changes in VUL MNs went undetected when analyzing whole laser-dissected ventral spinal cord (gray matter).

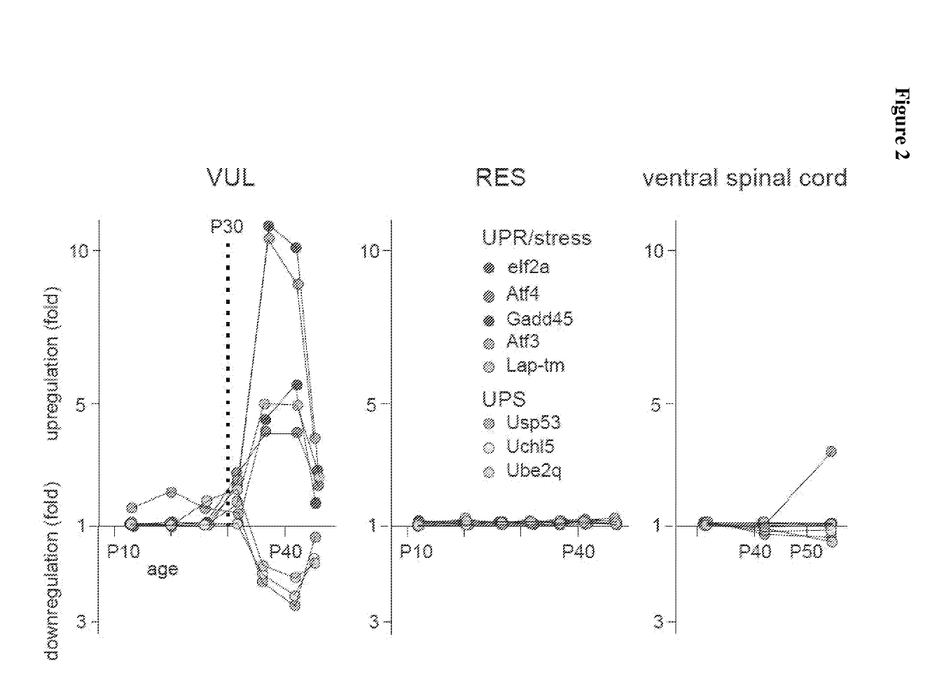

FIG. 3: Upregulation of UPR and stress genes, and downregulation of UPS genes in SOD1(G85R) VUL MNs from P130 on. The data for VUL MNs in SOD1(G93A) mice (graph on the left, identical to FIG. 2) are included for comparison.

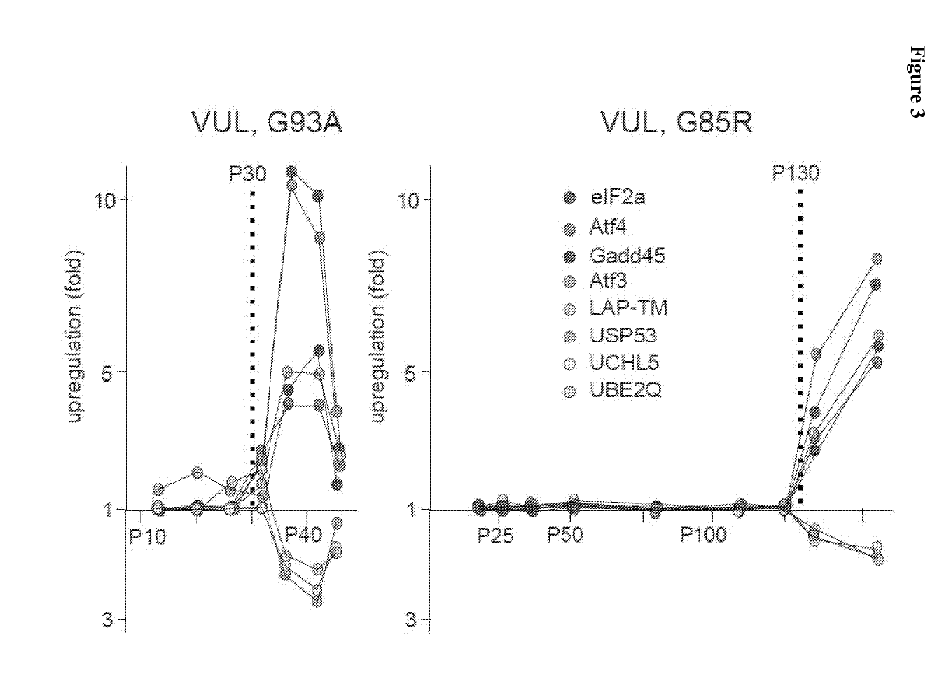

### DETAILED DESCRIPTION OF THE INVENTION

The present inventors have previously examined two mouse models of motoneuron (MN) disease (SOD1(G93A), SOD1/(G85R)), and found that axons of FF MNs are affected synchronously, long before symptoms; fast-fatigue-resistant MN axons are affected at the onset of symptoms, and axons of S MNs are resistant (Pun et al., 2006, Nature Neuroscience, 9(3):408-419).

One of the main aspects in understanding the initiation and progression of a neurodegenerative disease such as in ALS is to elucidate mechanisms that underlie or predispose a particular MN to selective vulnerability.

There is hence a need in the art for new reliable biomarkers that would be able to predict the imminent onset of a neurodegenerative disease.

In order to study gene expression alterations in MNs long before disease onset, at onset and during disease progression, the present inventors have selectively labeled vulnerable motoneurons innervating the purely FF subcompartment of lateral gastrocnemius, and resistant motoneurons innervating the soleus muscle at different times in wildtype and SOD (G93A) and G85R mice, followed by gene expression profiling of these selectively labeled 8-10 identified cells at a distinct time in disease. This approach has allowed the present inventors to investigate disease progression in defined vulnerable and resistant MNs in a “longitudinal” manner, i.e. comparing values at different times during the disease process.

The results obtained by the present inventors have surprisingly shown that 28 genes can be used as acute phase markers, whose alteration in expression specifically correlates with the imminent onset of macroscopically detectable degeneration first in vulnerable FF MNs, and later the FR MN. These genes were found to be up-regulated shortly before the onset of degeneration in both FF MNs, and FR MN, albeit at a later time point in said FR MN, which are known to degenerate later, and in both experimental murine models of the disease. This onset of degeneration rapidly leads to weakening of peripheral synapses, followed by peripheral axon degeneration (i.e. an active process of axon self-destruction) in vulnerable FF and FR MNs.

The present inventors therefore provide a method for the prediction of the imminent degeneration of motoneurons in a subject, said method comprising assessing in at least one motoneuron of said subject the expression of at least one gene selected from the group consisting of elF2a, Atf3, Laptm5, GADD45, LAPTM, BiP, Grn, and Nrn1, wherein an at least two-fold upregulation of the expression of the assessed gene is indicative of the imminent degeneration of motoneurons and/or of the imminent onset of a neurodegenerative disease. In one embodiment of the invention, the expression of at least one second gene selected from the group consisting of Hex B, Apeg1, Csf1r, Cth, Cyp3a11, Ddit4l, Gas5, Gbp2, Jag1, Kcnc2, Lzp-s, Nfe2l3, Ndrg1, Nrxn3, Pck2, Rex3, Slc7a3, Syt 7, Tyrobp, Zcchc12 and Zic1, is also assessed, wherein an at least two-fold upregulation of the expression of the assessed genes is indicative of the imminent degeneration of motoneurons and/or of the imminent onset of a neurodegenerative disease.

In one embodiment of the invention, the expression of at least Atf3, Laptm5, elF2a, GADD45, is assessed.

In some embodiments of the methods of the invention, an increase of the expression of at least one, two, three, four, five, six, seven or eight of these genes is indicative of the disease mediated stress response and imminent degeneration of motoneurons.

In yet another preferred embodiment of the invention, the expression of at least elF2a is assessed.

In another embodiment, the method of the invention further comprises assessing the presence of, or measuring the concentration of, a product of at least one gene selected from the group consisting of Ctss, C1qb, Igtb, Ifih1 and Tgfbi in a body fluid obtained from said subject.

In yet another embodiment of the method of the invention, said method further comprises assessing in at least one motoneuron of said subject the expression of at least one gene selected from the group consisting of Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd, wherein an at least two-fold downregulation of the expression of at least one of the assessed genes is indicative of the imminent degeneration of motoneurons and/or of the imminent onset of a neurodegenerative disease.

In some embodiments of the invention, the motoneurons of the subject present an accumulation of poly-ubiquitin, as compared to motoneurons of a control subject, or control population, and/or an increase in ribosomal protein S6 phosphorylation, as compared to motoneurons of a control subject, or control population.

Another aspect of the invention is the provision of a kit for predicting the imminent degeneration of motoneurons in a subject, said kit comprising means to assess in at least one motoneuron of said subject the expression of at least one gene selected from the group consisting of elF2a, Atf3, Laptm5, GADD45, LAPTM, BiP, Grn, Nrn1, Hex B, Apeg1, Csf1r, Cth, Cyp3a11, Ddit4l, Gas5, Gbp2, Jag1, Kcnc2, Lzp-s, Nfe2l3, Ndrg 1, Nrxn3, Pck2, Rex3, Slc7a3, Syt 7, Tyrobp, Zcchc12 and Zic1.

In an alternative embodiment of this aspect of the invention, the kit further comprises means to assess the presence and or concentration of a product of at least one gene selected from the group consisting of Ctss, C1qb, Igtb, Ifih1 and Tgfbi in a body fluid obtained from said subject.

In yet another embodiment of this aspect of the invention, the kit further comprises means to assess in at least one motoneuron of said subject the expression of at least one gene selected from the group consisting of Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd.

In an alternative invention, only the presence or concentration of at least one gene selected from the group consisting of Ctss, C1qb, Igtb, Ifih1 and Tgfbi is assessed in a body fluid obtained from said subject. This alternative also encompasses kits therefor. All the aspect of the present invention also applies for this alternative invention.

In yet another embodiment of the method of the invention, said method further comprises assessing in at least one motoneuron of said subject the expression of at least one gene selected from the group consisting of Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd, wherein an at least two-fold downregulation of the expression of at least one of the assessed genes is indicative of the imminent degeneration of motoneurons and/or of the imminent onset of a neurodegenerative disease.

In an alternative invention, only the expression of at least one gene selected from the group consisting of Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd is assessed in at least one motoneuron of the subject, wherein an at least two-fold downregulation of the expression of at least one of the assessed genes is indicative of the imminent degeneration of motoneurons and/or of the imminent onset of a neurodegenerative disease. This alternative also encompasses kits therefor. All the aspect of the present invention also applies for this alternative invention.

Another aspect of the invention is the provision of a kit for predicting the imminent degeneration of motoneurons in a subject, said kit comprising means to assess in at least one motoneuron of said subject the expression of at least one gene selected from the group consisting of Hex B, Apeg1, Atf3, Csf1r, Cth, Cyp3a11, Ddit4l, Gas5, Gbp2, Grn, Jag1, Kcnc2, Laptm5, Lzp-s, Nfe2l3, Ndrg 1, Nrn1, Nrxn3, Pck2, Rex3, Slc7a3, Syt 7, Tyrobp, Zcchc12 and Zic1.

In an alternative embodiment of this aspect of the invention, the kit further comprises means to assess the presence and or concentration of a product of at least one gene selected from the group consisting of Ctss, C1qb, Igtb, Ifih1 and Tgfbi in a body fluid obtained from said subject.

In yet another embodiment of this aspect of the invention, the kit further comprises means to assess in at least one motoneuron of said subject the expression of at least one gene selected from the group consisting of Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd.

A further alternative invention can be found in Table 3, which lists markers for the vulnerability of motoneurons to neurodegenerative diseases. All the aspect of the present invention also applies for this alternative invention.

The following tables list the genes of the invention. Unless otherwise indicated, the Entrez Gene GeneID number refers to the human gene (Homo sapiens). Depending on the species of the subject, the skilled person will be able to obtain without undue burden the corresponding sequence and particulars of the gene.

TABLE 1
Biomarkers for the onset of neurodegenerative diseases (up-regulated)

| Gene | | |
|---|---|---|
| name | Gene product | Entrez Gene GeneID |
| Hex B | Hexosaminidase B (beta polypeptide) | 3074 |
| Apeg1 | SPEG complex locus (Aortic preferentially expressed | 10290 |
| | protein 1) | |
| Atf3 | Activating transcription factor 3 | 467 |
| BiP | endoplasmic reticulum (ER) chaperone binding protein | |
| Csf1r | Colony stimulating factor 1 receptor | 1436 |
| Cth | Cystathionase (cystathionine gamma-lyase) | 1491 |
| Cyp3a11 | Cytochrome P450, family 3, subfamily a, polypeptide 11 | 13112 (M. musculus) |
| Ddit4l | DNA-damage-inducible transcript 4-like | 115265 |
| GADD45 | Growth arrest and DNA-damage-inducible gamma | |
| Gas5 | Growth arrest-specific 5 | 60674 |
| Gbp2 | Guanylate binding protein 2, interferon-inducible | 2634 |
| Grn | Granulin | 2896 |
| Jag1 | Jagged 1 (Alagille syndrome) | 182 |
| Kcnc2 | Potassium voltage-gated channel, Shaw-related subfamily, | 3747 |
| | member 2 | |
| LAPTM | lysosomal-associated protein transmembrane | |
| Laptm5 | Lysosomal associated multispanning membrane protein 5 | 7805 |
| Lzp-s | Lysozyme | 17105 (M. musculus) |
| Nfe2l3 | Nuclear factor (erythroid-derived 2)-like 3 | 9603 |
| Ndrg1 | N-myc downstream regulated gene 1 | 10397 |
| Nrn1 | Neuritin 1 | 51299 |
| Nrxn3 | Neurexin 3 | 9369 |
| Pck2 | Phosphoenolpyruvate carboxykinase 2 | 5106 |
| Rex3 | brain expressed (Bex1), X-linked 1; reduced expression 3 | 19716 (M. musculus) |
| Slc7a3 | Solute carrier family 7 (cationic amino acid transporter, y + | 84889 |
| | system), member 3 | |
| Syt 7 | Synaptotagmin VII | 9066 |
| Tyrobp | TYRO protein tyrosine kinase binding protein | 7305 |
| Zcchc12 | zinc finger, CCHC domain containing 12 | 170261 |
| Zic1 | Zic family member 1 (odd-paired homolog, Drosophila) | 7545 |

TABLE 2
Microglia-responsive genes correlating with
the onset of neurodegenerative diseases

| Gene | Gene product | Entrez Gene GeneID |
|---|---|---|
| Ctss | Cathepsin S | 1520 |
| C1qb | Complement component 1, q | 713 |
| | subcomponent, B chain | |
| Igtb | Interferon gamma induced GTPase | 16145 (M. musculus) |
| Ifih1 | Interferon induced with helicase | 64135 |
| | C domain 1 | |
| Tgfbi | Transforming growth factor, | 7045 |
| | beta-induced, 68 kDa | |

TABLE 3
Biomarkers for the onset of neurodegenerative diseases (down-regulated)

| Gene | | |
|---|---|---|
| name | Gene product | Entrez Gene GeneID |
| Car7 | Carbonic anhydrase 7 | 12354 (M. musculus) |
| Hist2h3c1 | Histone cluster 2, H3c1 | 15077 (M. musculus) |
| Tpmt | Thiopurine S-methyltransferase | 7172 |
| Igsf21 | Immunoglobin superfamily, member 21 | 84966 |
| Qars | Glutaminyl-tRNA synthetase | 5859 |
| Rab2b | RAB2B, member RAS oncogene family | 84932 |
| B3gnt6 | UDP-GlcNAc:betaGal beta-1,3-N- | 192134 |
| | acetylglucosaminyltransferase 6 | |
| Trhr | Thyrotropin-releasing hormone receptor | 7201 |
| Pop7 | Processing of precursor 7, ribonuclease P/MRP subunit | 10248 |
| | (S. cerevisiae) | |
| Bmp2k | BMP2 inducible kinase | 55589 |
| Scrn3 | Secernin 3 | 79634 |
| Atg7 | ATG7 autophagy related 7 homolog (S. cerevisiae) | 10533 |
| Eif3s1 | Eukaryotic translation initiation factor 3, subunit J | 8669 |
| Hip1r | Huntingtin interacting protein 1 related | 9026 |
| Siah1a | Seven in absentia 1A | 20437 (M. musculus) |
| Rab3b | RAB3B, member RAS oncogene family | 5865 |
| Slc18a3 | Solute carrier family 18 (vesicular acetylcholine), member 3 | 6572 |
| Rgs4 | Regulator of G-protein signaling 4 | 5999 |
| Stard4 | StAR-related lipid transfer (START) domain containing 4 | 134429 |
| E2f5 | E2F transcription factor 5, p130-binding | 1875 |
| Pik3cd | Phosphoinositide-3-kinase, catalytic, delta polypeptide | 5293 |
| USP53 | ubiquitin specific peptidase 53 | 54532 |
| UCHL5 | ubiquitin carboxyl-terminal esterase L5 | 51377 |
| UBE2Q | ubiquitin-conjugating enzyme E2Q family member 1 | 55585 |

TABLE 4

| Gene | Gene product | Entrez Gene GeneID |
|---|---|---|
| Limd1 | LIM domains containing 1 | 8994 |
| Sca7 | Ataxin 7 (spinocerebellar ataxia 7) | 6314 |
| Sox17 | SRY (sex determining region Y)-box 17 | 64321 |
| Tcf3 | Transcription factor 3 (E2A immunoglobulin enhancer | 6929 |
| | binding factors E12/E47) | |
| Ccrn4l | CCR4 carbon catabolite repression 4-like (S. cerevisiae) | 608468 |
| Slc6a6 | Solute carrier family 6 (neurotransmitter transporter, | 6533 |
| | taurine), member 6 | |
| Slc8a1 | Solute carrier family 8 (sodium/calcium exchanger), | 6546 |
| | member 1 | |
| Frmd4b | FERM domain containing 4B | 23150 |
| Phf10 | PHD finger protein 10 | 55274 |
| 2410006H16Rik | RIKEN cDNA 2410006H16 gene | 69221 (M. musculus) |
| Scg2 | Secretogranin II (chromogranin C) | 7857 |
| Efhd2 | EF-hand domain family, member D2 | 79180 |

The term “neurodegenerative disease” refers to a condition in which cells of the brain and spinal cord are lost. Examples of neurodegenerative diseases include, but are not limited to, Alexander disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Huntington disease, HIV-associated dementia, Kennedy's disease, Krabbe disease, Lewy body dementia, Machado-Joseph disease (Spinocerebellar ataxia type 3), Multiple sclerosis, Multiple System Atrophy, Neuroborreliosis, Parkinson disease, Pelizaeus-Merzbacher Disease, Pick's disease, Primary lateral sclerosis, Prion diseases, Refsum's disease, Sandhoff disease, Schilder's disease, Sub-Acute Combined Degeneration of the Cord Secondary to Pernicious Anaemia, Schizophrenia, Spielmeyer-Vogt-Sjogren-Batten disease (also known as Batten disease), Spinocerebellar ataxia (multiple types with varying characteristics), Spinal muscular atrophy, Steele-Richardson-Olszewski disease and Tabes dorsalis.

The term “motoneuron” or “motor neuron” applies to neurons located in the central nervous system (CNS) that project their axons outside the CNS and directly or indirectly control muscles. Motor neuron is also synonymous with efferent neuron. According to their targets, motoneurons are classified into three broad categories: “Somatic motoneurons”, which directly innervate skeletal muscles, involved in locomotion (such as muscles of the limbs, abdominal, and intercostal muscles), “Special visceral motoneurons”, also called “branchial motoneurons”, which directly innervate branchial muscles (that motorize the gills in fish and the face and neck in land vertebrates) and “General visceral motoneurons”, also termed “visceral motoneurons”, which indirectly innervate smooth muscles of the viscera (e.g. the heart, and the muscles of the arteries). Visceral motoneurons synapse onto neurons located in ganglia of the autonomic nervous system (sympathetic and parasympathetic), located in the peripheral nervous system (PNS), which themselves directly innervate visceral muscles (and also some gland cells). All motoneurons are cholinergic, i.e. they release the neurotransmitter acetylcholine. Parasympathetic ganglionic neurons are also cholinergic, whereas most sympathetic ganglionic neurons are noradrenergic, releasing the neurotransmitter noradrenaline. Somatic motoneurons are further subdivided into two types: alpha efferent neurons and gamma efferent neurons. “Alpha motoneurons” innervate extrafusal muscle fibers (also termed muscle fibers) located throughout the muscle. “Gamma motoneurons” innervate intrafusal muscle fibers found within the muscle spindle. In addition to voluntary skeletal muscle contraction, alpha motoneurons also contribute to muscle tone. Gamma motoneurons regulate the sensitivity of the spindle to muscle stretching.

Furthermore, alpha motoneurons can be further classified into the functional subtypes: fast-fatigable (FF), fast fatigue-resistant (FR) and slow (S) motoneurons, which show distinct excitability and recruitment properties and establish motor units (consisting of one motoneuron and all the muscle fibers it innervates) with markedly distinct fatigue and force properties (Burke, R. E. Physiology of motor units. in Myology (eds. Engel, A. G. & Franzini-Armstrong, C.) 464-484 (McGraw-Hill, New York, 1994)).

The term “motor neuron disease” or “motoneuron disease” comprises a group of severe disorders of the nervous system characterized by progressive degeneration of motor neurons (neurons are the basic nerve cells that combine to form nerves). Motor neurons control the behavior of muscles. Motor neuron diseases may affect the upper motor neurons, nerves that lead from the brain to the medulla (a part of the brain stem) or to the spinal cord, or the lower motor neurons, nerves that lead from the spinal cord to the muscles of the body, or both. Spasms and exaggerated reflexes indicate damage to the upper motor neurons. A progressive wasting (atrophy) and weakness of muscles that have lost their nerve supply indicate damage to the lower motor neurons. Examples of motor neuron diseases include, but are not limited to, Progressive Bulbar Palsy, Amyotrophic Lateral Sclerosis (ALS), Spinal Muscular Atrophy, Kugelberg-Welander Syndrome, Lou Gehrig's Disease, Duchenne's Paralysis, Werdnig-Hoffmann Disease, Juvenile Spinal Muscular Atrophy, Benign Focal Amyotrophy and Infantile Spinal Muscular Atrophy.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and mRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

As used herein, the term “genotype” means an unphased 5′ to 3′ sequence of nucleotide pair(s) found at one or more polymorphic sites in a locus on a pair of homologous chromosomes in an individual. As used herein, genotype includes a full-genotype and/or a sub-genotype.

As used herein, the term “locus” means a location on a chromosome or DNA molecule corresponding to a gene or a physical or phenotypic feature.

As used herein, the term “isogene” means the different forms of a given gene that exist in the population.

As used herein, the term “mutant” means any heritable variation from the wild-type that is the result of a mutation, e.g., single nucleotide polymorphism. The term “mutant” is used interchangeably with the terms “marker”, “biomarker”, and “target” throughout the specification.

As used herein, the term “nucleotide pair” means the nucleotides found at a polymorphic site on the two copies of a chromosome from an individual.

As used herein, the term “polymorphic site” means a position within a locus at which at least two alternative sequences are found in a population, the most frequent of which has a frequency of no more than 99%.

As used herein, the term “population” may be any group of at least two individuals. A population may include, e.g., but is not limited to, a reference population, a population group, a family population, a clinical population, and a same sex population.

As used herein, the term “phased” means, when applied to a sequence of nucleotide pairs for two or more polymorphic sites in a locus, the combination of nucleotides present at those polymorphic sites on a single copy of the locus is known.

As used herein, the term “polymorphism” means any sequence variant present at a frequency of >1% in a population. The sequence variant may be present at a frequency significantly greater than 1% such as 5% or 10% or more. Also, the term may be used to refer to the sequence variation observed in an individual at a polymorphic site. Polymorphisms include nucleotide substitutions, insertions, deletions and microsatellites and may, but need not, result in detectable differences in gene expression or protein function.

As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified e.g. for stability or for other reasons.

As used herein, the term “polypeptide” means any polypeptide comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well-known in the art. Such modifications are well described in basic texts and in more detailed monographs, as well as in a voluminous research literature.

As used herein, the term “reference standard population” means a population characterized by one or more biological characteristics, e.g., drug responsiveness, genotype, haplotype, phenotype, etc.

As used herein, the term “reference standard gene expression profile” is the pattern of expression of one or more gene observed in either a reference standard population or a single subject prior to administration of a compound.

As used herein, the term “subject” means that preferably the subject is a mammal, such as a human, but can also be an animal, including but not limited to, domestic animals (e.g., dogs, cats and the like), farm animals (e.g., cows, sheep, pigs, horses and the like) and laboratory animals (e.g., monkeys such as cynmologous monkeys, rats, mice, guinea pigs and the like).

As used herein, a “test sample” means a biological sample obtained from a subject of interest. For example, a test sample can be a biological fluid (e.g., serum), cell sample, or tissue, or isolated nucleic acid or polypeptide derived therefrom.

As used herein, the expression “body fluid” is a biological fluid selected from a group comprising blood, bile, blood plasma, serum, aqueous humor, amniotic fluid, cerebrospinal fluid, sebum, intestinal juice, semen, sputum, sweat and urine.

As used herein, the term “dysregulation” means a change that is larger or equal to 1.2 fold and statistically significant (p<0.05, Student's t-test) from the control. For example, a 1.5, 2, 2.5, 3, 3.5, 4, 4.5, or 5 fold change.

“Upregulation” and “downregulation” of gene expression can be assessed using statistical methods well known to the person skilled in the art.

As used herein, “imminent” means that the onset of an event, e.g. neurodegeneration, will happen at the latest within five years. It is however to be understood that this term is relative and depends, for example, of the nature of the subject. In certain subjects, e.g. mice, this timeline will be much shorter. Alternatively, imminent neurodegenerative events can already have started, without however showing a phenotype yet.

### Amplifying a Target Gene Region.

The target region(s) may be amplified using any oligonucleotide-directed amplification method, including but not limited to polymerase chain reaction (PCR). (U.S. Pat. No. 4,965,188), ligase chain reaction (LCR) (Barany et al., Proc. Natl. Acad. Sci. USA, 88:189-193 (1991); published PCT patent application WO 90/01069), and oligonucleotide ligation assay (OLA) (Landegren et al., Science, 241:1077-1080 (1988)). Oligonucleotides useful as primers or probes in such methods should specifically hybridize to a region of the nucleic acid that contains or is adjacent to the polymorphic site. Other known nucleic acid amplification procedures may be used to amplify the target region including transcription-based amplification systems. (U.S. Pat. No. 5,130,238; EP 0 329 822; U.S. Pat. No. 5,169,766, published PCT patent application WO 89/06700) and isothermal methods (Walker et al., Proc. Natl. Acad. Sci., USA, 89:392-396 (1992).

### Hybridizing Specifc Oligonucleotide to a Target Gene.

Hybridization of a specifc oligonucleotide to a target polynucleotide may be performed with both entities in solution, or such hybridization may be performed when either the oligonucleotide or the target polynucleotide is covalently or noncovalently affixed to a solid support. Attachment may be mediated, for example, by antibody-antigen interactions, poly-L-Lysine, streptavidin or avidin-biotin, salt bridges, hydrophobic interactions, chemical linkages, UV cross-linking, baking, etc. Specifc oligonucleotide may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibres, chips, dishes, and beads. The solid support may be treated, coated or derivatised to facilitate the immobilization of the specifc oligonucleotide or target nucleic acid. The genotype or haplotype for the gene of an individual may also be determined by hybridization of a nucleic sample containing one or both copies of the gene to nucleic acid arrays and subarrays such as described in WO 95/11995. The arrays would contain a battery of specifc oligonucleotides representing each of the polymorphic sites to be included in the genotype or haplotype. See, also, Molecular Cloning A Laboratory Manual, Second Ed., Sambrook, Fritsch & Maniatis, ed. (Cold Spring Harbor Laboratory Press, 1989); DNA Cloning, Volumes I and II, Glover D N ed. (1985); Oligonucleotide Synthesis, Gait M J ed. (1984); Nucleic Acid Hybridization, Hames B D & Higgins S J, eds., 1984).

### Computer System for Storing or Displaying Gene Expression or Polymorphism Data.

The invention also provides a computer system for storing and displaying data determined for the gene. Polymorphism data is information that includes, but is not limited to, e.g., the location of polymorphic sites; sequence variation at those sites; frequency of polymorphisms in one or more populations; the different genotypes and/or haplotypes determined for the gene; frequency of one or more of these genotypes and/or haplotypes in one or more populations; any known association(s) between a trait and a genotype or a haplotype for the gene. The computer system comprises a computer processing unit, a display, and a database containing the polymorphism data. The polymorphism data includes the polymorphisms, the genotypes and the haplotypes identified for a given gene in a reference population. In a preferred embodiment, the computer system is capable of producing a display showing gene expression pattern organized according to their evolutionary relationships. In addition, the computer may execute a program that generates views (or screens) displayed on a display device and with which the user can interact to view and analyze large amounts of information, relating to the gene and its genomic variation, including chromosome location, gene structure, and gene family, gene expression data, polymorphism data, genetic sequence data, and clinical data population data (e.g., data on ethnogeographic origin, clinical responses, and gene expression pattern for one or more populations). The polymorphism data described herein maybe stored as part of a relational database (e.g., an instance of an Oracle database or a set of ASCII flat files). These polymorphism data may be stored on the computer's hard drive or may, for example, be stored on a CD-ROM or on one or more other storage devices accessible by the computer. For example, the data may be stored on one or more databases in communication with the computer via a network.

### Kits of the Invention.

It is to be understood that the methods of the invention described herein generally may further comprise the use of a kit according to the invention. The invention provides nucleic acid and polypeptide detection kits useful for assessing the expression of the genes of the invention in an individual. Such kits are useful to classify subjects. Generally, the methods of the invention may be performed ex-vivo, and such ex-vivo methods are specifically contemplated by the present invention. Also, where a method of the invention may include steps that may be practised on the human or animal body, methods that only comprise those steps which are not practised on the human or animal body are specifically contemplated by the present invention.

The kits of the invention are useful for detecting the presence of a polypeptide or nucleic acid corresponding to a marker of the invention in a biological sample, e.g., any body fluid including, but not limited to, e.g., serum, plasma, lymph, cystic fluid, urine, stool, cerebrospinal fluid, acidic fluid or blood and including biopsy samples of body tissue. For example, the kit can comprise a labelled compound or agent capable of detecting a polypeptide or an mRNA encoding a polypeptide corresponding to a marker of the invention in a biological sample and means for determining the amount of the polypeptide or mRNA in the sample e.g., an antibody which binds the polypeptide or an oligonucleotide probe which binds to DNA or mRNA encoding the polypeptide.

For antibody-based kits, the kit can comprise, e.g., (1) a first antibody, e.g., attached to a solid support, which binds to a polypeptide corresponding to a marker or the invention; and, optionally; (2) a second, different antibody which binds to either the polypeptide or the first antibody and is conjugated to a detectable label.

For oligonucleotide-based kits, the kit can comprise, e.g., (1) an oligonucleotide, e.g., a detectably-labelled oligonucleotide, which hybridizes to a nucleic acid sequence encoding a polypeptide corresponding to a marker of the invention; or (2) a pair of primers useful for amplifying a nucleic acid molecule corresponding to a marker of the invention.

The kit can also comprise, e.g., a buffering agent, a preservative or a protein-stabilizing agent. The kit can further comprise components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. In a preferred embodiment, such kit may further comprise a DNA sample collecting means. The kits of the invention may contain a written product on or in the kit container. The written product describes how to use the reagents contained in the kit, e.g., to use the biomarkers of the present invention in determining a strategy for preventing or treating a medical condition in a subject In several embodiments, the use of the reagents can be according to the methods of the invention. In one embodiment, the reagent is a gene chip for determining the gene expression of relevant genes.

### Detection of Biomarker Gene Expression.

An exemplary method for detecting the presence or absence of a polypeptide or nucleic acid in a biological sample involves obtaining a biological sample from a test subject and contacting the biological sample with a compound, or a compound capable of detecting polypeptide or nucleic acid (e.g., mRNA, genomic DNA) that encodes polypeptide of interest, such that the presence of gene is detected in the biological sample. A compound for detecting mRNA or genomic DNA is a labelled nucleic acid probe capable of hybridizing to mRNA or genomic DNA. The nucleic acid probe can be, for example, a full-length nucleic acid or a portion thereof, such as an oligonucleotide of at least 5, 15, 30, 50, 100, 250 or 500 nucleotides in length and sufficient to specifically hybridize under stringent conditions to mRNA or genomic DNA. Other suitable probes for use in the diagnostic assays of the invention are described herein. An example of a compound for detecting a polypeptide of the invention is an antibody raised against a specific polypeptide, capable of binding to the specific polypeptide, preferably an antibody with a detectable label. Antibodies can be polyclonal, or more preferably, monoclonal. An intact antibody, or a fragment thereof (e.g., Fab or F(ab′)₂) can be used. The term “labelled”, with regard to the probe or antibody, is intended to encompass direct labelling of the probe or antibody by coupling (i.e., physically linking) a detectable substance to the probe or antibody, as well as indirect labelling of the probe or antibody by reactivity with another compound that is directly labelled. Examples of indirect labelling include detection of a primary antibody using a fluorescently-labelled secondary antibody and end-labelling of a DNA probe with biotin such that it can be detected with fluorescently-labelled streptavidin. That is, the detection method of the invention can be used to detect mRNA, polypeptide, or genomic DNA of the invention in a biological sample in vitro as well as in vivo. For example, in vitro techniques for detection of mRNA include Northern hybridizations and in situ hybridizations. In vitro techniques for detection of polypeptide of the invention include enzyme linked immunosorbent assays (ELISAs), Western blots, immunoprecipitations, and immunofluorescence. In vitro techniques for detection of genomic DNA include Southern hybridizations. Furthermore, in vivo techniques for detection of polypeptide include introducing into a subject a labelled anti-polypeptide antibody. For example, the antibody can be labelled with a radioactive marker whose presence and location in a subject can be detected by standard imaging techniques. In one embodiment, the biological sample contains polypeptide molecules from the test subject. Alternatively, the biological sample can contain mRNA molecules from the test subject or genomic DNA molecules from the test subject.

In practicing the present invention, many conventional techniques in molecular biology, protein biochemistry, cell biology, immunology, microbiology and recombinant DNA can be used. These techniques are well-known and are explained in, e.g., Current Protocols in Molecular Biology, Vols. I-III, Ausubel, Ed. (1997); Sambrook et al., Molecular Cloning: A Laboratory Manual, Second Edition (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, New York, 1987); and Meth. Enzymol., Vols. 154 and 155, Wu & Grossman, and Wu, eds., respectively. Methods to detect and measure mRNA levels (i.e., gene transcription level) and levels of polypeptide gene expression products (i.e., gene translation level) are well-known in the art and include the use of nucleotide microarrays and polypeptide detection methods involving mass spectrometers and/or antibody detection and quantification techniques. See also, Strachan & Read, Human Molecular Genetics, Second Edition. (John Wiley and Sons, Inc., New York, 1999).

Techniques for the detection of gene expression of the genes described by this invention include, but are not limited to Northern blots, RT-PCT, real time PCR, primer extension, RNase protection, RNA expression profiling and related techniques. Techniques for the detection of gene expression by detection of the protein products encoded by the genes described by this invention include, but are not limited to, e.g., antibodies recognizing the protein products, western blots, immunofluorescence, immunoprecipitation, ELISAs and related techniques. These techniques are well known to those of skill in the art. Sambrook J. et al., Molecular Cloning: A Laboratory Manual, Third Edition (Cold Spring Harbor Press, Cold Spring Harbor, N.Y., 2000). In one embodiment, the technique for detecting gene expression includes the use of a gene chip. The construction and use of gene chips are well known in the art. See, U.S. Pat. Nos. 5,202,231; 5,445,934; 5,525,464; 5,695,940; 5,744,305; 5,795,716 and 5,800,992. See also, Johnston M, Curr. Biol., 8:R171-174 (1998); Iyer V R et al., Science, 283:83-87 (1999) and Elias P, “New human genome ‘chip’ is a revolution in the offing” Los Angeles Daily News (Oct. 3, 2003).

### Determination of Biomarker Gene Translation.

In another embodiment of the present invention, a polypeptide corresponding to a marker is detected. The detection of the biomarker polypeptide (a.k.a., biomarker, marker, marker protein or marker polypeptide) expression product of the biomarker gene in body fluids or tissues can be used to determine the presence or absence of the polymorphism, and the relative level of the biomarker polypeptide expression product can be used to determine if the polymorphism is present in a homozygous or heterozygous state (and hence the risk category of the individual). That is, in another embodiment of the present invention, a polypeptide corresponding to a marker (i.e., biomarker polypeptide) is detected. The level of this biomarker polypeptide gene expression product in body fluids or tissue sample may be determined by any means known in the art.

### Immunological Detection Methods.

Expression of the protein encoded by the gene(s) of the invention can be detected by a probe which is detectably labelled, or which can be subsequently labelled. Generally, the probe is an antibody that recognizes the expressed protein. A variety of formats can be employed to determine whether a sample contains a biomarker protein that binds to a given antibody. Immunoassay methods useful in the detection of biomarker polypeptides of the present invention include, but are not limited to, e.g., dot blotting, western blotting, protein chips, competitive and non-competitive protein binding assays, enzyme-linked immunosorbant assays (ELISA), immunohistochemistry, fluorescence activated cell sorting (FACS), and others commonly used and widely-described in scientific and patent literature, and many employed commercially. A skilled artisan can readily adapt known protein/antibody detection methods for use in determining whether cells express a marker of the present invention and the relative concentration of that specific polypeptide expression product in blood or other body tissues. Proteins from individuals can be isolated using techniques that are well-known to those of skill in the art. The protein isolation methods employed can, e.g., be such as those described in Harlow & Lane, Antibodies: A Laboratory Manual (Cold Spring Harbor, N.Y., 1988)). An intact antibody, or a fragment thereof, e.g., Fab or F(ab′)₂ can be used. Antibody fragments, which recognize specific epitopes, may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (see Huse et al., Science, 246:1275-1281 (1989)), to allow rapid and easy identification of monoclonal Fab fragments with the desired specificity. The term “labelled”, with regard to the probe or antibody, is intended to encompass direct-labelling of the probe or antibody by coupling, i.e., physically linking, a detectable substance to the probe or antibody, as well as indirect-labelling of the probe or antibody by reactivity with another reagent that is directly-labelled. Examples of indirect labelling include detection of a primary antibody using a fluorescently-labelled secondary antibody and end-labelling of a DNA probe with biotin such that it can be detected with fluorescently-labelled streptavidin.

Monoclonal antibodies (mAbs), which are homogeneous populations of antibodies to a particular antigen, may be obtained by any technique that provides for the production of antibody molecules by continuous cell lines in culture. These include, but are not limited to, the hybridoma technique of Kohler & Milstein, Nature, 256:495-497 (1975); and U.S. Pat. No. 4,376,110; the human B-cell hybridoma technique of Kosbor et al., Immunol. Today, 4:72 (1983); Cole et al., Proc. Natl. Acad. Sci., USA, 80:2026-2030 (1983); and the EBV-hybridoma technique, Cole et al., Monoclonal Antibodies and Cancer Therapy, pp. 77-96 (Alan R. Liss, Inc., 1985). Such antibodies may be of any immunoglobulin class including IgG, IgM, IgE, IgA, IgG and any subclass thereof. The hybridoma producing the mAb of this invention may be cultivated in vitro or in vivo. Production of high titres of mAbs in vivo makes this the presently preferred method of production.

In addition, techniques developed for the production of “chimaeric antibodies” (see Morrison et al., Proc. Natl. Acad. Sci. USA, 81:6851-6855 (1984); Neuberger et al., Nature, 312: 604-608 (1984); and Takeda et al., Nature, 314:452-454 (1985)), by splicing the genes from a mouse antibody molecule of appropriate antigen specificity together with genes from a human antibody molecule of appropriate biological activity can be used. A chimaeric antibody is a molecule in which different portions are derived from different animal species, such as those having a variable or hypervariable region derived form a murine mAb and a human immunoglobulin constant region.

Alternatively, techniques described for the production of single chain antibodies, U.S. Pat. No. 4,946,778; Bird, Science, 242:423-426 (1988); Huston et al., Proc. Natl. Acad. Sci. USA, 85:5879-5883 (1988); and Ward et al., Nature, 334:544-546 (1989), can be adapted to produce differentially expressed gene single-chain antibodies. Single-chain antibodies are formed by linking the heavy- and light-chain fragments of the Fv region via an amino acid bridge, resulting in a single-chain polypeptide.

More preferably, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429. Antibody fragments, which recognize specific epitopes, may be generated by known techniques. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (see Huse et al., Science, 246:1275-1281 (1989)), to allow rapid aid easy identification of monoclonal Fab fragments with the desired specificity. In one format, antibodies or antibody fragments can be used in methods, such as Western blots or immunofluorescence techniques, to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros and magnetite.

The extent to which the known proteins are expressed in a biological sample is determined by immunoassay methods that utilize the antibodies described above. A typical example is the sandwich ELISA, of which a number of variations exist, all of which are intended to be used in the methods and assays of the present invention. For example, in a typical forward assay, unlabeled antibody is immobilized on a solid substrate and the sample to be tested brought into contact with the bound molecule after a suitable period of incubation, for a period of time sufficient to allow formation of an antibody-antigen binary complex. At this point, a second antibody, labelled with a reporter molecule capable of inducing a detectable signal, is then added and incubated, allowing time sufficient for the formation of a ternary complex of antibody-antigen-labelled antibody. Any unreacted material is washed away, and the presence of the antigen is determined by observation of a signal, or may be quantitated by comparing with a control sample containing known amounts of antigen. Variations on the forward assay include the simultaneous assay, in which both sample and antibody are added simultaneously to the bound antibody, or a reverse assay in which the labelled antibody and sample to be tested are first combined, incubated and added to the unlabelled surface bound antibody. These techniques are well-known to those skilled in the art, and the possibility of minor variations will be readily apparent. As used herein, “sandwich assay” is intended to encompass all variations on the basic two-site technique. For the immunoassays of the present invention, the only limiting factor is that the labelled antibody must be an antibody that is specific for the protein expressed by the gene of interest.

### Two-Dimensional Gel Electrophoresis.

Proteins can be separated by two-dimensional gel electrophoresis systems and then identified and/or quantified. Two-dimensional gel electrophoresis is well-known in the art and typically involves isoelectric focusing along a first dimension followed by SDS PAGE electrophoresis along a second dimension. (See, e.g., Hames et al., Gel Electrophoresis of Proteins: A Practical Approach (IRL Press, NY, 1990); Shevchenko et al., Proc Natl. Acad. Sci. USA, 93:14440-14445 (1996); Sagliocco et al., Yeast, 12:1519-1533 (1996); and Lander, Science 274: 536-539 (1996)). The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells, e.g., in yeast, exposed to a drug, or in cells modified by, e.g., deletion or over-expression of a specific gene.

### Mass Spectroscopy.

The identity and the expression level of biomarker polypeptide can both be determined using mass spectroscopy technique (MS). MS-based analysis methodology is use for analysis of isolated biomarker polypeptide as well as analysis of biomarker polypeptide in a biological sample. MS formats for use in analyzing a biomarker polypeptide include ionization (I) techniques, such as, but not limited to, MALDI, continuous or pulsed ESI and related methods, such as ionspray or thermospray, and massive cluster impact (MCI). Such ion sources can be matched with detection formats, including linear or non-linear reflectron TOF, single or multiple quadrupole, single or multiple magnetic sector, Fourier transform ion cyclotron resonance (FTICR), ion trap and combinations thereof such as ion-trap/TOF. For ionization, numerous matrix/wavelength combinations (MALDI) or solvent combinations (ESI) can be employed. Sub-attomole levels of protein have been detected, e.g., using ESI MS (Valaskovic et al., Science, 273:1199-1202 (1996)) and MALDI MS (Li et al., J. Am. Chem. Soc., 118:1662-1663 (1996)).

For MS analysis, the biomarker polypeptide can be solubilised in an appropriate solution or reagent system. The selection of a solution or reagent system, e.g., an organic or inorganic solvent, will depend on the properties of the biomarker polypeptide and the type of MS performed, and is based on methods well-known in the art. See, e.g., Vorm et al., Anal. Chem., 61:3281 (1994) for MALDI; and Valaskovic et al., Anal. Chem., 67:3802 (1995), for ESI. MS of peptides also is described, e.g., in International PCT Application No. WO 93/24834 and U.S. Pat. No. 5,792,664. A solvent is selected that minimizes the risk that the biomarker polypeptide will be decomposed by the energy introduced for the vaporization process. A reduced risk of biomarker polypeptide decomposition can be achieved, e.g., by embedding the sample in a matrix. A suitable matrix can be an organic compound such as a sugar, e.g., a pentose or hexose, or a polysaccharide such as cellulose. Such compounds are decomposed thermolytically into CO₂ and H₂O such that no residues are formed that can lead to chemical reactions. The matrix can also be an inorganic compound, such as nitrate of ammonium, which is decomposed essentially without leaving any residue. Use of these and other solvents is known to those of skill in the art. See, e.g., U.S. Pat. No. 5,062,935. Electrospray MS has been described by Fenn et al., J. Phys. Chem., 88:4451-4459 (1984); and in PCT Application No. WO 90/14148; and current applications are summarized in review articles. See Smith et al., Anal. Chem., 62:882-89 (1990); and Ardrey, Spectroscopy, 4:10-18 (1992). With ESI, the determination of molecular weights in femtomole amounts of sample is very accurate due to the presence of multiple ion peaks, all of which can be used for mass calculation.

Matrix Assisted Laser Desorption (MALDI) is one preferred method among the MS methods herein. Methods for performing MALDI are well-known to those of skill in the art. Numerous methods for improving resolution are also known. For example, resolution in MALDI-TOF-MS can be improved by reducing the number of high energy collisions during ion extraction. See, e.g., Juhasz et al., Analysis, Anal. Chem., 68:941-946 (1996); see also, e.g., U.S. Pat. Nos. 5,777,325; 5,742,049; 5,654,545; 5,641,959; 5,654,545, and 5,760,393 for descriptions of MALDI and delayed extraction protocols. MALDI-TOF: MS has been described by Hillenkamp et al., Burlingame & McCloskey, eds., pp. 49-60 (Elsevier Science Publ., 1990). In a preferred embodiment, the level of the biomarker protein in a biological sample, e.g., body fluid or tissue sample, maybe measured by means of mass spectrometric (MS) methods including, but not limited to, those techniques known in the art as matrix-assisted laser desorption/ionization, time-of-flight mass spectrometry (MALDI-TOF-MS) and surfaces enhanced for laser desorption/ionization, time-of-flight mass spectrometry (SELDI-TOF-MS) as further detailed below.

### MASLDI-TOF-MS Protein Detection Technique.

In some preferred embodiments, the detection of specific proteins or polypeptide gene expression products in a biological sample, e.g., body fluid or tissue sample, is performed by means of MS, especially matrix-assisted laser desorption/ionization, time-of-flight mass spectrometry (MASLDI-TOF-MS). These techniques have been used to analyze macromolecules, such as proteins or biomolecules and utilize sample probe surface chemistries that enable the selective capture and desorption of analytes, including intact macromolecules, directly from the probe surface into the gas (vapour phase), and in the most preferred embodiments without added chemical matrix.

In other embodiments a variety of other techniques for marker detection using mass spectroscopy can be used. See Bordeaux Mass Spectrometry Conference Report, Hillenkamp, ed., pp. 354-362 (1988); Bordeaux Mass Spectrometry Conference Report, Karas & Hillenkamp, Eds., pp. 416-417 (1988); Karas & Hillenkamp, Anal. Chem., 60:2299-2301 (1988); and Karas et al., Biomed Environ Mass Spectrum, 18:841-843 (1989). The use of laser beams in TOF-MS is shown, e.g., in U.S. Pat. Nos. 4,694,167; 4,686,366; 4,295,046; and 5,045,694, which are incorporated herein by reference in their entireties. Other MS techniques allow the successful volatilization of high molecular weight biopolymers, without fragmentation, and have enabled a wide variety of biological macromolecules to be analyzed by mass spectrometry.

### Surfaces Enhanced for Laser Desorption/Ionization (SELDI).

In a preferred embodiment of the present invention, other techniques are used which employ new MS probe element compositions with surfaces that allow the probe element to actively participate in the capture and docking of specific analytes, described as Affinity Mass Spectrometry (AMS). Several types of new MS probe elements have been designed with Surfaces Enhanced for Affinity Capture (SEAC). See Hutchens & Yip, Rapid Commun. Mass Spectrom., 7:576-580 (1993). SEAC probe elements have been used successfully to retrieve and tether different classes of biopolymers, particularly proteins, by exploiting what is known about protein surface structures and biospecific molecular recognition.

In another preferred embodiment of the present invention, the method of detection to be used with the methods of this invention uses a general category of probe elements, i.e., sample presenting means with surfaces enhanced for laser desorption/ionization (SELDI). See SELDI U.S. Pat. Nos. 5,719,060; 5,894,063; 6,020,208; 6,027,942; 6,124,137; and US. Patent Application No. U.S. 2003/0003465.

A polypeptide of interest can be attached directly to a support via a linker. Any linkers known to those of skill in the art to be suitable for linking peptides or amino acids to supports, either directly or via a spacer, may be used. For example, the polypeptide can be conjugated to a support, such as a bead, through means of a variable spacer. Linkers, include, Rink amide linkers (see, e.g., Rink, Tetrahedron Lett., 28:3787 (1976)); trityl chloride linkers (see, e.g., Leznoff, Ace Chem. Res. 11:327 (1978)); and Merrifield linkers. (See, e.g., Bodansky et al., Peptide Synthesis, Second Edition (Academic Press, New York, 1976)). For example, trityl linkers are known. (See, e.g., U.S. Pat. Nos. 5,410,068 and 5,612,474). Amino trityl linkers are also known, (See, e.g., U.S. Pat. No. 5,198,531). Other linkers include those that can be incorporated into fusion proteins and expressed in a host cell. Such linkers may be selected amino acids, enzyme substrates or any suitable peptide. The linker may be made, e.g., by appropriate selection of primers when isolating the nucleic acid. Alternatively, they may be added by post-translational modification of the protein of interest.

As developed herein-above, expression of genes listed in the Tables may be investigated by analysis of target molecules representative of gene expression in the sample. The presence or absence of target molecules in a sample will generally be detected using suitable probe molecules. Such detection will provide information as to gene expression, and thereby allow comparison between gene expression occurring in the subject and expression occurring in the control sample. Probes will generally be capable of binding specifically to target molecules directly or indirectly representative of gene expression. Binding of such probes may then be assessed and correlated with gene expression to allow an effective prognostic comparison between gene expression in the subject and in the control.

As used herein, the expression “altered expression” includes where the gene expression is both elevated or reduced in the sample when compared to the control, as discussed above.

Conversely “unaltered expression” includes where the gene expression is not elevated or reduced in the sample when compared to the control, as discussed above.

An assessment of whether a gene expression is altered or unaltered can be made using routine methods of statistical analysis.

As explained above, the target molecule may be peptide or polypeptide. Preferably the amount of peptide or polypeptide is determined using a specific binding molecule, most preferably an antibody. In a preferred instance, the amount of certain target proteins present in a sample may be assessed with reference to the biological activity of the target protein in the sample. Assessment and comparison of expression in this manner is particularly suitable in the case of protein targets having enzyme activity. Suitable techniques for the measurement of the amount of a protein target present in a sample include, but are not limited to, aptamers and antibody-based techniques, such as radio-immunoassays (RIAs), enzyme-linked immunoassays (ELISAs) and Western blotting.

Nucleic acids represent preferred target molecules for assaying gene expression according to the invention.

For the avoidance of doubt, it is to be understood that “nucleic acids” or “nucleic acid molecules” for the purposes of the present invention refer to deoxyribonucleotide or ribonucleotide polymers in either single- or double-stranded form. Furthermore, unless the context requires otherwise, these terms should be taken to encompass known analogues of natural nucleotides that can function in a similar manner to naturally occurring nucleotides.

Furthermore it will be understood that target nucleic acids suitable for use in accordance with the invention need not comprise “full length” nucleic acids (e.g. full length gene transcripts), but need merely comprise a sufficient length to allow specific binding of probe molecules.

It is preferred that the nucleic acid target molecule is a mRNA gene transcript and artificial products of such transcripts. Preferred examples of artificial target molecules generated from gene transcripts include cDNA and cRNA, either of which may be generated using well known protocols or commercially available kits or reagents.

In a preferred embodiment of the method of the invention, samples may be treated to isolate RNA target molecules by a process of lysing cells taken from a suitable sample (which may be achieved using a commercially available lysis buffer such as that produced by Qiagen Ltd.) followed by centrifugation of the lysate using a commercially available nucleic acid separation column (such as the RNeasy midi spin column produced by Qiagen Ltd). Other methods for RNA extraction include variations on the phenol and guanidine isothiocyanate method of Chomczynski, P. and Sacchi, N. (1987) Analytical Biochemistry 162, 156. “Single Step Method of RNA Isolation by Acid Guanidinium Thiocyanate-Phenol-Chloroform Extraction.” RNA obtained in this manner may constitute a suitable target molecule itself, or may serve as a template for the production of target molecules representative of gene expression.

It may be preferred that RNA derived from a subject or control sample may be used as substrate for cDNA synthesis, for example using the Superscript System (Invitrogen Corp.). The resulting cDNA may then be converted to biotinylated cRNA using the BioArray RNA Transcript labelling Kit (Enzo Life Sciences Inc.) and this cRNA purified from the reaction mixture using an RNeasy mini kit (Qiagen Ltd).

mRNA, representative of gene expression, may be measured directly in a tissue derived from a subject or control sample, without the need for mRNA extraction or purification. For example, mRNA present in, and representative of gene expression in, a subject or control sample of interest may be investigated using appropriately fixed sections or biopsies of such a tissue. The use of samples of this kind may provide benefits in terms of the rapidity with which comparisons of expression can be made, as well as the relatively cheap and simple tissue processing that may be used to produce the sample. In situ hybridisation techniques represent preferred methods by which gene expression may be investigated and compared in tissue samples of this kind. Techniques for the processing of tissues of interest that maintain the availability of RNA representative of gene expression in the subject or control sample are well known to those of skill in the art.

However, techniques by which mRNAs representative of gene expression in a subject or control sample may be extracted and collected are also well known to those skilled in the art, and the inventors have found that such techniques may be advantageously employed in accordance with the present invention. Samples comprising extracted mRNA from a subject or control sample may be preferred for use in the method of the invention, since such extracts tend to be more readily investigated than is the case for samples comprising the original tissues. For example, suitable target molecules allowing for comparison of gene expression may comprise the total RNA isolated from a sample of tissue from the subject, or a sample of control tissue.

Furthermore, extracted RNA may be readily amplified to produce an enlarged mRNA sample capable of yielding increased information on gene expression in the subject or control sample. Suitable examples of techniques for the extraction and amplification of mRNA populations are well known, and are considered in more detail herein-above and below.

By way of example, methods of isolation and purification of nucleic acids to produce nucleic acid targets suitable for use in accordance with the invention are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993).

In a preferred method, the total nucleic acid may be isolated from a given sample using, the techniques described in the Example.

In the event that it is desired to amplify the nucleic acid targets prior to investigation and comparison of gene expression it may be preferred to use a method that maintains or controls for the relative frequencies of the amplified nucleic acids in the subject or control tissue from which the sample is derived.

Suitable methods of “quantitative” amplification are well known to those of skill in the art. One well known example, quantitative PCR, involves simultaneously co-amplifying a control sequence whose quantities are known to be unchanged between control and subject samples. This provides an internal standard that may be used to calibrate the PCR reaction.

In addition to the methods outlined above, the skilled person will appreciate that any technology coupling the amplification of gene-transcript specific product to the generation of a signal may also be suitable for quantitation. A preferred example employs convenient improvements to the polymerase chain reaction (U.S. Pat. Nos. 4,683,195 and 4,683,202) that have rendered it suitable for the exact quantitation of specific mRNA transcripts by incorporating an initial reverse transcription of mRNA to cDNA. Further key improvements enable the measurement of accumulating PCR products in real-time as the reaction progresses.

In many cases it may be preferred to assess the degree of gene expression in subject or control samples using probe molecules capable of indicating the presence of target molecules (representative of one or more of the genes of the invention) in the relevant sample.

Probes for use in the method of the invention may be selected with reference to the product (direct or indirect) of gene expression to be investigated. Examples of suitable probes include oligonucleotide probes, antibodies, aptamers, and binding proteins or small molecules having suitable specificity.

Oligonucleotide probes constitute preferred probes suitable for use in accordance with the method of the invention. The generation of suitable oligonucleotide probes is well known to those skilled in the art (Oligonucleotide synthesis: Methods and Applications, Piet Herdewijn (ed) Humana Press (2004).). Oligonucleotide and modified oligonucleotides are commercially available from numerous companies.

For the purposes of the present invention an oligonucleotide probe may be taken to comprise an oligonucleotide capable of hybridising specifically to a nucleic acid target molecule of complementary sequence through one or more types of chemical bond. Such binding may usually occur through complementary base pairing, and usually through hydrogen bond formation. Suitable oligonucleotide probes may include natural (ie., A, G, C, or T) or modified bases (7-deazaguanosine, inosine, etc.). In addition, a linkage other than a phosphodiester bond may be used to join the bases in the oligonucleotide probe(s), so long as this variation does not interfere with hybridisation of the oligonucleotide probe to its target. Thus, oligonucleotide probes suitable for use in the methods of the invention may be peptide nucleic acids in which the constituent bases are joined by peptide bonds rather than phosphodiester linkages.

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA). Preferably a probe may bind, duplex or hybridise only to the particular target molecule.

The term “stringent conditions” refers to conditions under which a probe will hybridise to its target subsequence, but minimally to other sequences. Preferably a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Preferred technologies permit the parallel quantitation of the expression of multiple genes and include technologies where amplification and quantitation of species are coupled in real-time, such as the quantitative reverse transcription PCR technologies and technologies where quantitation of amplified species occurs subsequent to amplification, such as array technologies.

Array technologies involve the hybridisation of samples, representative of gene expression within the subject or control sample, with a plurality of oligonucleotide probes wherein each probe preferentially hybridises to a disclosed gene or genes. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonucleotide arrays may be synthesised in situ (e.g by light directed synthesis as commercially provided by Affymetrix Inc) or pre-formed and spotted by contact or ink-jet technology (as commercially provided by Agilent or Applied Biosystems). It will be apparent to those skilled in the art that whole or partial cDNA sequences may also serve as probes for array technology (as commercially provided by Clontech).

Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane.

In certain circumstances the use of traditional hybridisation protocols for comparing gene expression may prove problematic. For example blotting techniques may have difficulty distinguishing between two or more gene products of approximately the same molecular weight since such similarly sized products are difficult to separate using gels. Accordingly, in such circumstances it may be preferred to compare gene expression using alternative techniques, such as those described below.

Gene expression in a sample representing gene expression in a subject may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes selected from those listed above and in Table 2. Accordingly it will be recognised that such oligonucleotide arrays may be particularly preferred in embodiments of the methods of the invention where it is desired to compare expression of more than one gene of the invention.

Other suitable methodologies that may be used in the comparison of nucleic acid targets representative of gene expression include, but are not limited to, nucleic acid sequence based amplification (NASBA); or rolling circle DNA amplification (RCA).

It is usually desirable to label probes in order that they may be easily detected. Examples of detectable moieties that may be used in the labelling of probes or targets suitable for use in accordance with the invention include any composition detectable by spectroscopic, photochemical, biochemical, immunochemical, electrical, optical or chemical means. Suitable detectable moieties include various enzymes, prosthetic groups, fluorescent materials, luminescent materials, bioluminescent materials, radioactive materials and colourimetric materials. These detectable moieties are suitable for incorporation in all types of probes or targets that may be used in the methods of the invention unless indicated to the contrary.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, phycoerythrin, texas red, rhodamine, green fluorescent protein, and the like; an example of a luminescent material includes luminol; examples of bioluminescent materials include luciferase, luciferin, and aequorin; examples of suitable radioactive material include ¹²⁵I, ¹³¹I, ³⁵S, ³H, ¹⁴C, or ³²P; examples of suitable colorimetric materials include colloidal gold or coloured glass or plastic (e.g., polystyrene, polypropylene, latex, etc.) beads.

Means of detecting such labels are well known to the skilled person. For example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the coloured label.

In a preferred embodiment of the invention fluorescently labelled probes or targets may be scanned and fluorescence detected using a laser confocal scanner.

In the case of labelled nucleic acid probes or targets suitable labelling may take place before, during, or after hybridisation. In a preferred embodiment, nucleic acid probes or targets for use in the methods of the invention are labelled before hybridisation. Fluorescence labels are particularly preferred and, where used, quantification of the hybridisation of the nucleic acid probes to their nucleic acid targets is by quantification of fluorescence from the hybridised fluorescently labelled nucleic acid. More preferably quantitation may be from a fluorescently labelled reagent that binds a hapten incorporated into the nucleic acid.

In a preferred embodiment of the invention analysis of hybridisation may be achieved using suitable analysis software, such as the Microarray Analysis Suite (Affymetrix Inc.).

Effective quantification may be achieved using a fluorescence microscope which can be equipped with an automated stage to permit automatic scanning of the array, and which can be equipped with a data acquisition system for the automated measurement, recording and subsequent processing of the fluorescence intensity information. Suitable arrangements for such automation are conventional and well known to those skilled in the art.

In a preferred embodiment, the hybridised nucleic acids are detected by detecting one or more detectable moieties attached to the nucleic acids. The detectable moieties may be incorporated by any of a number of means well known to those of skill in the art. However, in a preferred embodiment, such moieties are simultaneously incorporated during an amplification step in the preparation of the sample nucleic acids (probes or targets). Thus, for example, polymerase chain reaction (PCR) using primers or nucleotides labelled with a detectable moiety will provide an amplification product labelled with said moiety. In a preferred embodiment, transcription amplification using a fluorescently labelled nucleotide (e.g. fluorescein-labelled UTP and/or CTP) incorporates the label into the transcribed nucleic acids.

Alternatively, a suitable detectable moiety may be added directly to the original nucleic acid sample (e.g., mRNA, polyA mRNA, cDNA, etc. from the tissue of interest) or to an amplification product after amplification of the original nucleic acid is completed. Means of attaching labels such as fluorescent labels to nucleic acids are well known to those skilled in the art and include, for example nick translation or end-labelling (e.g. with a labeled RNA) by kinasing of the nucleic acid and subsequent attachment (ligation) of a nucleic acid linker joining the sample nucleic acid to a label (such as a suitable fluorophore).

The expression “axon-protecting factors” refers to factors protecting motoneurons from neurodegenerative diseases. The expression “axon-protecting factors” includes neutrophic factors. Neurotrophic factors have been suggested as potential therapeutic agents for motor neuron diseases (Thoenen et al., Exp. Neurology 124,47-55, 1993). Indeed, embryonic motor neuron survival in culture is enhanced by members of the neurotrophin family such as brain derived neurotrophic factor (BDNF), neurotrophin-3 (NT-3), NT-4 (NT-4/5), cytokines such as ciliary neurotrophic factor (CNTF), leukaemia inhibitory factor (LIF) and cardiotrophin-1, glial cell line-derived neurotrophic factor (GDNF), insulin-like growth factor-1 (IGF-1) and members of the FGF family (review in Henderson, Neurotrophic factors as therapeutic agents in amyotrophic lateral sclerosis: potential and pitfalls. In Serratrice G. T. and Munsat T. L. eds. Pathogenesis and therapy of amyotrophic lateral sclerosis. Advances in Neurology, 68, pp. 235-240, 1995. Lippincott-Raven publishers, Philadelphia; Pennica et al., Cardiotrophin-1, a cytokine present in embryonic muscle, supports long-term survival of spinal motoneurons. Neuron, 17, 63-74, 1996). In vivo, a reduction of motoneuronal death occurring naturally during embryonic development was observed with CNTF (Oppenheim et al., Control of embryonic motoneuron survival in vivo by ciliary neurotrophic factor. Science, 251, 1616-1618, 1991), BDNF (Oppenheim et al., Brain-derived neurotrophic factor rescues developing avian motoneurons from cell death. Nature, 360, 755-757, 1992), GDNF (Oppenheim et al., Developing motor neurons rescued from programmed and axotomy-induced cell death by GDNF. Nature, 373, 344-346, 1995), and cardiotrophin-1 (Pennica et al., 1996). Protection from retrograde motor neuron death after acute peripheral nerve axotomy in neonate rodents was evidenced with several factors (Sendtner et al., Ciliary neurotroptuc factor prevents the degeneration of motor neurons after axotomy, Nature 345, 440-441, 1990, Sendtner et al., Ciliary neurotrophic factor prevents degeneration of motor neurons in mouse mutant progressive motor neuronopathy. Nature, 358, 502-504, 1992; Sendtner et al., Brain-derived neurotrophic prevents the death of motoneurons in newborn rats after nerve section. Nature, 360, 757-759, 1992; Vejsada et al., Quantitative comparison of the transient rescue effects of neurotrophic factors on axotomised motoneurons in vivo. Eur. J. Neurosci., 7, 108-115, 1995). Also, a protective effect of CNTF and/or BDNF was described in two murine models of inherited progressive motor degeneration (Sendtner et al., 1992; Mitsumoto et al., Arrest of motor neuron disease in wobbler mice cotreated with CNTF and BDNF. Science, 265, 1107-1110, 1994). The preferred neurotrophic factors are ciliary neurotrophic factor (CNTF), glial cell maturation factors (GMFa, b), GDNF, BDNF, NT-3, NT-5 and the like. The neurotrophic factor NT-3 is particularly preferred. The complete nucleotide sequence encoding NT-3 is disclosed in WO91/03569, the contents of which are incorporated herein by reference.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

### Materials & Methods

Retrograde labeling of Motoneurons

Mice were anaesthetized using a 1:1.5 μg/gm body weight mix of ketamine and rompun (xylazine, PROVET AG, Switzerland), and maintained at 36.9° C. during the surgical process. Hind limbs were epilated, and an incision was made into the skin, to expose the muscle of interest. RITC-Dextrane (Invitrogen Corp., Carlsbad, Calif. 92008) was either 3,000 MW (D-3308) or 10,000 MW (fluororuby D-1817, lysine fixable), and was injected locally (6 mg/ml in PBS-0.1% BSA; 5-10 μl volume) into lateral gastrocnemius (subcompartment-I1) or soleus muscle. The incision was sutured using Silkam® USP 6 (B. Braun Medical AG, Switzerland). Where indicated, CNTF rat recombinant protein (Sigma Aldrich) was reconstituted at a concentration of 1 μg/ml in 0.1% PBS-BSA, and triceps surae muscle was injected every alternate day with 80-100 μl of the working solution. Salubrinal (Alexis Biochemicals) was reconstituted at a concentration of 2.6 mM in PBS, 0.1% BSA, with 10% DMSO, and triceps surae muscle was injected every day (P14-P21) or every alternate day (P22-P32; P39-P50) with 80-100 μl of the working solution.

Mice were sacrificed 2-3 days after tracer application (4 days for mice older than P50). Spinal cords were then rapidly collected, embedded in Tissue-Tek OCT Compound (Zoeterwoude, The Netherlands), placed at −20° C. for 1 h, and then stored at −80° C. Upon cryostat sectioning (14 μm), all sections were mounted on membrane slides (MMI, Glattbrugg, Switzerland), and dehydrated in 95% ethanol for 15 s. Upon air-drying, single motoneurons were immediately microdissected using a MMI CellCut laser dissection microscope (MMI, Glattbrugg, Switzerland). Criteria for motoneuron selection included a diameter of greater than 20 μm and an identifiable nucleus. Approximately 25-30 cell sections, representing 10-11 cells, were collected per tube.

### RNA Isolation, Amplification and Microarray Profiling

Laser-dissected cells were immediately covered with 50 μl extraction buffer (Arcturus, USA), left to lyse on the lid of the tube at RT for at least 15 minutes, and stored at −80° C. Total cellular RNA was isolated with the PicoPure RNA isolation kit (Arcturus, USA), with minor modifications. The optional DNase treatment step to remove potential genomic DNA contamination was omitted and the centrifugation times during washing were reduced to 30 seconds.

All RNA samples were processed in batches of maximally 4 times three replicates (12 samples in total). Preparation of in vitro transcription (IVT) products was performed according to Affymetrix protocols (GeneChip Expression Analysis Technical manual, Rev. 5), with minor modifications. The fragmented cRNA (15 μg) was used in a hybridization cocktail containing spiked controls (Affymetrix). A total of 200 μl of this hybridization cocktail was hybridized at 45° C. for 16 hours to GeneChip Mouse Genome 430 2.0 Arrays (Affymetrix). Following hybridization, the arrays were processed using a GeneChip Fluidics Station 400 according to recommended protocols (FS450_{—}0001, Affymetrix). Fluorescent images of arrays were captured using the GeneChip Scanner 3000 7G (Affymetrix), and image data were acquired and analyzed using the GeneChip Operating Software (GCOS, Affymetrix). Average present call values were about 50%.

### Statistical Analysis

Expression values and detection p-values were calculated using the GC-RMA implementation found in Genedata's (Basel, Switzerland) Refiner array 4.1 package. Expression values were quantile normalized and median scaled to a value of 20 in Genedata's Analyst 4.1 package. Gene expression changes were evaluated upon removal of any gene with a detection p-value >0.04 (Absent) in all conditions.

A statistical power analysis was performed to determine the minimum fold difference we could consider with a power of 0.8 or better. Based on this, the inventors found that their experiment was able to reliably detect fold changes greater than 1.5. To further reduce the number of genes in the analysis, the inventors performed either a 1.5 or 2 fold filter on the genes present in at least one condition and constructed a union list of these genes (this reduces the false discovery implications of subsequent statistical analyses). This list was then used to identify statistically significant effects in the data set using an N-way ANOVA (p<0.01). False discovery rate was estimated using a Benjamini and Hochberg correction. Genes passing these filters were then subjected to SOM clustering and pathway analysis using Ingenuity's Pathway Suite.

### Immunocytochemistry

Mice were perfused with 4% PFA, lumbar level 3-5 (L3-L5) spinal cord was isolated, and kept overnight at 4° C. in 30% sucrose. After embedding, 50 μm cryostat sections were post fixed with 4% PFA for 10 min, followed by PBS washes. Antibodies used for immunocytochemistry were: rabbit-anti Ero1-Lα, 1:20 (Cell Signaling Technology, Inc, Danvers, Mass. 01923), mouse-anti KDEL Grp78, Grp94 (BiP), 1:500 (Stressgen, Ann Arbor, Mich. 48108), rabbit-anti VAchT, 1:1000 (Chemicon International, Temecula, Calif.), rat-anti CD11b, 1:200 (Serotec, Kidlington, OX5 1GE), rabbit-anti ATF3 (H90), 1:200 (Santa Cruz Biotechnology, Inc, Santa Cruz, Calif. 95060), rabbit-anti-synaptophysin, 1:200 (Dako, Glostrup, DK), and rabbit-anti Pi-elF2α (Ser 51) 1:100 (Cell Signaling Technology). Antibodies were applied in PBS-3% BSA-0.3% Triton X 100, and incubated overnight at 4° C. Sections were then briefly washed with PBS and incubated for 60 min at RT with goat-anti-rabbit (Alexa 488, Invitrogen) or goat-anti-mouse (Alexa 546, Invitrogen). ATF3 stainings were done on freshly embedded 50 μm cryostat tissue sections fixed with 4% PFA for 10 min. Pi-elF2α stainings were carried out on 50 μm floating sections in the presence of 1 mM Na-Vanadate. For muscle labelings, sections were processed as described above, and acetylcholine receptors at the synapse were visualized with FITC-α-Bungarotoxin (1:1000; Invitrogen). Confocal images were acquired using an Olympus Fluoview (Olympus, Tokyo) microscope, fitted with a 20× air objective, or an LSM Meta (Carl Zeiss A G) microscope, fitted with a 40× oil objective. Images were processed using Imaris software.

For the analysis of BiP (or Pi-elF2α) labeling intensities, data were acquired with identical confocal settings, ensuring that signals at the brightest cells were not saturated, and that background levels outside clusters were still detectable. Images were then analyzed quantitatively using Image-Pro 5 software (Media Cybernetics). Signal intensities were acquired within areas inside cells, excluding areas lacking signal; background levels outside cells were subtracted from these values.

### Results

### Longitudinal Gene Profiling of Identified Neurons in Genetic Model of MN Disease

To generate gene expression profiles from identified VUL and RES MNs derived from individual adult mice, the inventors applied the retrograde tracer rhodamine-dextran locally into the most lateral subcompartment of lateral gastrocnemius muscle (innervated by 11 VUL MNs) or into soleus muscle (innervated by 21 RES MNs), and, 2 days later, collected 10-11 retrogradely labeled VUL or RES MNs using laser dissection microscopy. RNA was subjected to two rounds of amplification, followed by hybridization to mouse Affymetrix gene arrays. The inventors found that for more than 14,000 detectable gene probes on the array, average raw expression values in the subtypes of MNs exhibited standard errors lower than 10%, with a majority below the 5% range (N=3 mice). These mean raw expression values were highly reproducible among adult mice of different ages or genotype, and genes whose expression values did change between two different ages exhibited similar age-dependences among different genotypes. These findings thus demonstrate the feasibility of longitudinal gene profiling for the same small groups of identified VUL and RES MNs in mice of different genotypes. The subsequent analysis revealed that the approach was highly effective to detect early disease-related alterations selectively in VUL MNs, which went undetected in parallel experiments involving laser dissection of whole ventral spinal cord.

### Increasing ER Stress Specifically in VUL MNs Leading Up to Disease Onset

A detailed analysis of cells selectively compromised or spared by disease at an age preceding axonal pathology in VUL MNs revealed that all cell types included in the analysis exhibited substantial numbers of altered transcripts in mutant mice. This finding likely reflected ubiquitous expression of mutant SOD1(G93A) in the transgenic mice and highlighted the challenge of identifying gene alterations specifically related to the disease process.

Since alterations in gene expression per se did not seem predictive of vulnerability early in disease, the inventors determined whether a longitudinal analysis of vulnerable MNs might uncover changes indicative of disease-relevant responses in these neurons. The inventors found that from P12 on, which for technical reasons was the earliest age that could be included in their gene profiling analysis, VUL (but not RES) MNs of SOD1(G93A) mice specifically exhibited an upregulation of genes involved in stress-related pathways, including protein ubiquitination, hypoxia and NRF2-mediated stress pathways. This stress-related response increased up to P26, and was lost abruptly at P32 and beyond. The loss of early stress-related responses was followed by an upregulation of UPR genes and a downregulation of Ubiquitin Proteasome System (UPS) genes in VUL MNs. These vigorous cellular stress responses were detectable at P32 but not at P26, and peaked at P38. In parallel to the UPR, VUL MNs specifically upregulated typical stress markers, such as Atf3. Immunocytochemistry with ATF3 antibodies confirmed specific upregulation in retrogradely labeled VUL but not RES MNs from P32 onwards, validating the gene profiling results. Validation was also obtained for the stress-induced genes Laptm5, Granulin, Neuritin1 and Hexosaminidase B.

To determine whether the onset of a UPR in VUL MNs might define disease onset in the MN disease mice, the inventors analyzed the distribution and morphology of spinal cord microglia, i.e. local microenvironment cells that have been causally linked to disease progression in MN disease. The inventors found that in SOD1(G93A) mice CD11b-positive microglia underwent a rapid and dramatic morphological change and size expansion between P28 and P32 in lumbar ventral spinal cord. The activation of microglia was accompanied by triggering of inflammatory genes specifically in VUL MNs. The longitudinal analysis of stress responses selectively in VUL MNs thus revealed a sharp transition associated with local microglia activation around P30, which likely reflects disease onset in this model of MN disease.

To investigate the relationship between cellular stress pathways and selective vulnerability in MN disease in more detail, the inventors labeled spinal cord sections with antibodies against the lumenal ER proteins BiP and ERO1-Lα, whose upregulation is diagnostic of increasing ER stress. The inventors found that from the earliest age included in our analysis (P5) a subset of lumbar spinal cord MNs exhibited elevated BiP immunoreactivity, that those BiP signals increased gradually up to P28, and that they then rose more abruptly at P32 and beyond. The inventors then labeled spinal cord sections with an antibody against phosphorylated eukaryotic Initiation Factor 2α (Pi-elF2α) which reveals the molecular point of convergence of cellular stress sensors, to initiate comprehensive adaptive responses in stressed cells, including a UPR16,17. The inventors detected elevated Pi-elF2a signals in the same cells exhibiting elevated BiP signals, where the Pi-elF2α response only became detectable at P28, and then increased markedly up to P38. A detailed analysis of lumbar spinal cord sections double-labeled for BiP and the MN marker Vesicular Acetylcholine Transporter (VAChT) revealed that while BiP signal intensities per individual overexpressing MN increased substantially with age, constant fractions of MNs overexpressed BiP between P20 and P38. BiP-overexpressing MN fractions in lumbar (level L4) spinal cord were 47-50%, and consistent with estimated VUL MN contents in the corresponding motor pools. Focusing on the triceps pool within the caudal half of lumbar segment L423, BiP-overexpressing MNs fractions were in good agreement with expected VUL MN contents (detected 48%, expected 48-52%). Consistent with a one-to-one relationship between BiP overexpressing MNs and VUL MNs, immunocytochemistry of retrogradely labeled MNs revealed that at P28 all VUL MNs innervating subcompartment-I1 of lateral gastrocnemius overexpressed BiP, whereas no soleus RES MN did. These results thus support the notion that in SOD1(G93A) mice lumbar VUL MNs selectively exhibit increasing BiP immunoreactivity diagnostic of ER stress from soon after birth on, and Pi-elF2a immunoreactivity coupled to markedly elevated BiP immunoreactivity diagnostic of a UPR, from P28-30 onwards.

### VUL MNs are Selectively Prone to Stress

To determine whether VUL MNs might be generally more prone to stress than RES MNs, the inventors carried out unilateral sciatic nerve crush experiments in wild-type and SOD1(G93A) mice at P24, and analyzed spinal cords for BiP immunoreactivity 1-4 days later. The crush-regeneration process is associated with a dramatic reorganization of the ER and of protein synthesis in these neurons. The inventors found that a fraction of MNs exhibited elevated BiP signals upon peripheral nerve crush, and that the magnitude of these fractions were closely comparable in wild-type and SOD1(G93A) mice. Significantly, while the crush procedure produced strongly elevated BiP signals in individual MNs of SOD1(G93A) mice, it did not affect the fraction of MNs with elevated BiP signals in those mice, suggesting that even in the presence of excess SOD1(G93A), RES MNs remained more resistant to this axonal stress procedure. In support of this conclusion, experiments involving retrograde labeling followed by local nerve crush confirmed that VUL MNs further upregulated BiP upon nerve crush in SOD1(G93A) mice, whereas RES MNs did not. These results thus provide evidence that VUL MNs are selectively prone to stress, suggesting that this fact might underlie their selective vulnerability in MN disease mice.

Comparable ER Stress Progression Selectively in VUL MNs in Two Disease Models with Distinct Kinetics

To investigate whether VUL MNs might also be affected selectively by increasing ER stress in a mutant SOD1 model of MN disease with delayed disease onset, the inventors determined whether a similar sequence of characteristic ER stress states restricted to VUL MNs might underlie the disease process in SOD1(G85R) mice. They found that in these mice a fraction of lumbar MNs exhibited increasing BiP signal levels between P20 and P160. Consistent with a later disease onset, elevated BiP signals at P30 and P80 were lower than those detected in SOD1(G93A) mice at P20, and at P26. Significantly, fractions of MNs with elevated BiP signals were constant between P20 and P130, and comparable to those detected in SOD1(G93A) mice. Again, elevated BiP signals were detected in all VUL MNs innervating subcompartment-I1 lateral gastrocnemius, but in no soleus RES MNs. In further striking analogy to SOD1(G93A) mice, the inventors detected an abrupt increase in ER stress at P130 in the SOD1(G85R) model (i.e., again, 20-30 days before peripheral denervation by VUL MNs), which was accompanied by the upregulation of the characteristic UPR and stress genes specifically in VUL MNs, and by the activation of microglia in lumbar spinal cord. Therefore, the slower disease process in SOD1(G85R) mice is mainly reflected by a slower increase in VUL MN BiP signals before disease onset at P130, which is followed by more comparable disease progression processes in the two mutant SOD1 models.

### Axon Pathology is Preceded by a Comparable Sequence of ER Stress Processes in VUL and RES MNs

To determine whether increasing ER stress consistently anticipates axonal pathology in MN disease, the inventors determined whether RES MNs might undergo comparable cellular stress responses before their peripheral denervation in mutant SOD1 mice. A detailed analysis of SOD1(G93A) lumbar spinal sections revealed a second substantial subpopulation of MNs with slightly elevated BiP signals at P38 but not at P32. After P50, retrograde labeling from mixed VUL/RES subcompartments of lateral gastrocnemius muscle allowed them to selectively label RES MNs due to complete pruning of peripheral innervation by VUL MNs6. The gene profiling analysis revealed that lateral gastrocnemius RES MNs expressed stress-related genes in patterns resembling those in VUL MNs, but with a delay of 25-30 days. This amounted to a UPR onset for RES MNs at about P55, i.e. 25-35 days prior to their peripheral denervation. Closely comparable results were obtained when retrogradely labeled soleus MNs (no VUL MNs innervating this muscle) were analyzed, ruling out the possibility that the alterations in lateral gastrocnemius RES MNs were due to sprouting and reinnervation of synapses that had been vacated by VUL MNs. Further supporting the notion that the disease progression phases in the SOD1(G93A) and SOD1(G85R) models are comparable, we made analogous observations in SOD1(G85R) mice, where the UPR onset for RES MNs was around P160.

### CNTF-sensitive ER Stress in VUL MNs Determines Disease Onset and Progression

To determine whether the increasing ER stress responses in VUL MNs were causally related to the disease process, the inventors enhanced or reduced ER stress in SOD1(G93A) mice, and monitored the influence of these treatments on disease onset and progression. In a first series of experiments, the inventors enhanced stress in VUL MNs by crushing the sciatic nerve, which contains the axons of MNs innervating the hindlimb, at P24 (i.e. about 6 days before disease onset). In wildtype mice, this nerve crush procedure enhanced VUL MN BiP signals, but had no detectable effect on MN Pi-elF2a immunoreactivity or on spinal cord CD11b-positive microglia. In contrast, in SOD1(G93A) mice the peripheral nerve lesion induced a marked and progressive upregulation of Pi-elf2a immunoreactivity in about 50% of spinal MNs. Significantly, the peripheral lesion was accompanied by a dramatic expansion of CD11b-positive microglia, which initially accumulated in the vicinity of Pi-elF2α-positive MNs. These results provided evidence that enhanced axonal stress in VUL MNs accelerates the onset of a UPR, and triggers microglia activation in MN disease mice.

In a second series of experiments, the inventors determined whether the stress responses in SOD1(G93A) mice were affected by intramuscular applications of the specific stress-protecting factor CNTF, a treatment that effectively attenuates axonal pathology in VUL MNs and slows disease progression in these mice. The inventors found that applying CNTF from P20 to P28, i.e. before disease onset, completely prevented the upregulation of BiP in VUL MNs. Furthermore, applying CNTF from P28 to P40 effectively prevented the UPR/UPS reaction, normalized VUL MN BiP and Pi-elF2a signals, counteracted the downregulation of stress-related protective pathways in VUL MNs, and reduced the activation of microglia on the spinal cord side ipsilateral to the treatment. The sequence of early stress-related responses selectively in VUL MNs and the selective axonal pathology later in disease, thus appear to be part of the same CNTF-sensitive disease-determining process in these MN disease mice.

### Salubrinal Arrests the Disease Process

To investigate in more detail how promoting stress coping interferes with the disease process, and to define the therapeutic potential of drugs that promote stress coping in MN disease, the inventors delivered the inhibitor of Pi-elF2a dephosphorylation Salubrinal into one (ipsilateral) hindlimb, and vehicle into the contralateral limb. In one set of experiments, the inventors applied Salubrinal from P14 (i.e. 16 days before disease onset) until P32, and analyzed the mice 7 days (P39) and 21 days (P53) after discontinuation of the treatment. The inventors found that the early Salubrinal treatment led to low levels of BiP, no evidence of a UPR, and no microglia activation at P39, and to early signs of a UPR, mild microglia activation, and no loss of synaptic vesicles at neuromuscular junctions at P53. These results suggested that Salubrinal had effectively arrested the disease process during its administration, and that the process had resumed from a low level upon drug removal. In a second set of experiments, the inventors applied Salubrinal from P39 (i.e. 9 days after disease onset) until P50, and analyzed the mice 2 days later (P53). They found that the late Salubrinal treatment led to a mild UPR, mild microglia activation, and no loss of neuromuscular junction synaptic vesicles at P53. Taken together, these results suggested that the disease process had been partially reversed during the Salubrinal treatments, and that it resumed from a lower level upon drug removal. The results further suggested that the status of disease progression at any particular time might be the result of a time-dependent disease process driven by ER stress, rather than an absolute function of the animal's age.

As is apparent to one of ordinary skill in the art, variations in the above-described methods can be introduced with ease to attain the same objective. Various incubating conditions, labels, apparatus and materials can be chosen according to individual preference. All publications referred to herein are incorporated by reference in their entirety as if each were referred to individually.

1. A method for predicting the imminent degeneration of motoneurons in a subject, said method comprising
assessing in at least one motoneuron of said subject the expression of at least one gene selected from the group consisting of elF2a, Atf3, Laptm5, GADD45, LAPTM, BiP, Grn, and Nrn1,
wherein an at least two-fold upregulation of the expression of the assessed gene is indicative of the imminent degeneration of motoneurons and/or of the imminent onset of a neurodegenerative disease.

2. The method of claim 1 wherein the expression of at least one second gene selected from the group consisting of Hex B, Apeg1, Csf1r, Cth, Cyp3a11, Ddit4l, Gas5, Gbp2, Jag1, Kcnc2, Lzp-s, Nfe2l3, Ndrg1, Nrxn3, Pck2, Rex3, Slc7a3, Syt 7, Tyrobp, Zcchc12 and Zic1,
wherein an at least two-fold upregulation of the expression of the assessed genes is indicative of the imminent degeneration of motoneurons and/or of the imminent onset of a neurodegenerative disease.

3. The method of claim 1 wherein the expression of at least Atf3, Laptm5, elF2a, GADD45, is assessed.

4. The method of claim 1 wherein an at least two-fold increase of the expression of at least two, three, four, five, six, seven or eight of the assessed genes is indicative of the imminent degeneration of motoneurons and/or of the imminent onset of a neurodegenerative disease.

5. The method of claim 1 wherein at least the expression of at least elF2a is assessed.

6. The method of claim 1 further comprising assessing the presence or measuring the concentration of a product of at least one gene selected from the group consisting of Ctss, C1qb, Igtb, Ifih1 and Tgfbi in a body fluid obtained from said subject.

7. The method of claim 1 further comprising assessing in at least one motoneuron of said subject the expression of at least one further gene selected from the group consisting of USP53, UCHL5, UBE2Q, Car7, Hist2h3c1, Tpmt, Igsf21, Qars, Rab2b, B3gnt6, Trhr, Pop7, Bmp2k, Scrn3, Atg7, Eif3s1, Hip1r, Siah1a, Rab3b, Slc18a3, Rgs4, Stard4, E2f5, Pik3cd wherein an at least two-fold downregulation of the expression of at least one of the assessed further genes is indicative of the imminent degeneration of motoneurons and/or of the imminent onset of a neurodegenerative disease.

8. The method of claim 1 wherein the motoneurons of the subject present an accumulation of poly-ubiquitin, as compared to motoneurons of a control subject, or control population.

9. The method of claim 1 wherein the motoneurons of the subject present an increase in ribosomal protein S6 phosphorylation, as compared to motoneurons of a control subject, or control population.
